(19) United States
(12) Patent Application Publication
(10) Pub. No.: US 2022/0379047 A1
(43) Pub. Date: Dec. 1, 2022

(54) Spray Device and Force Control System

(71) Applicants: Medspray Anesthesia B.V., Enschede (NL); Medspray B.V., Enschede (NL)

(72) Inventors: Marcus Alexander Van Barneveld, Enschede (NL); Jorgen Bruhn, Enschede (NL); Geert-Jan Van Geffen, Enschede (NL); Wilhelmus Petrus Johannes De Kruijf, Enschede (NL)

(21) Appl. No.: 17/772,635

(22) PCT Filed: Nov. 4, 2020
(86) PCT No.: PCT/IB2020/060376

§ 371 Date: Apr. 28, 2022

(30) Foreign Application Priority Data
Nov. 4, 2019 (NL) 1043447

(51) Int. Cl. A61M 11/00

(52) U.S. Cl. CPC A61M 11/007; A61M 11/008; A61M 2210/065

(57) ABSTRACT

A spray device includes a mouthpiece that accommodates a spray nozzle unit. The spray nozzle unit is operatively connected to a liquid reservoir to receive a pressurized liquid to be sprayed. A connection between the spray nozzle unit and the liquid reservoir is effected by a flexible tube that connects at one end substantially fixedly to the mouth piece in fluid communication with the spray nozzle device. The liquid reservoir is interchangeably coupled to the opposite end of the flexible tube and, preferably, comprises a standard syringe. A spray delivery device comprises a holder for holding the syringe and has at least one manipulator member that is configured to impose an enhanced force on the plunger.

The present invention relates to a spray device for the oral administration of a therapeutic, in particular pharmaceutical, active agent, comprising a liquid container for holding an amount of a liquid to be nebulized that comprises the active agent and comprising an inhalation member, comprising a mouthpiece that is intended and adapted to be received orally by a user, wherein said inhalation member is spatially separated from the container and is coupled to the container via a flexible fluid line, wherein said mouthpiece comprises a spray nozzle unit which is configured and adapted to receive the liquid to be nebulized under an elevated operating pressure and to create a mist therefrom containing the active agent. The present invention moreover relates to a force control system.

The present invention particularly relates to a disposable soft mist spray device for topical anaesthetic drug delivery to the patient's mouth, throat and/or respiratory tract. The device may be used to increase patient comfort in preparation for an intubation, a bronchoscopy or an upper-gastroscopy.

A device for oral administration of a pharmaceutical agent is for instance known from US 2015/0165136. This known drug delivery device comprises a flexible elongate shaft having a number of radially opening spray orifices at an end portion and a spray nozzle unit connected to the distal end of said elongate shaft. A syringe may be connected to a proximal end of this spray head in order to deliver a selected medicament though said spray head both radially and distally into the oral cavity.

Such device is typically intended for the local delivery of an anaesthetic, like lidocaine, to the larynx and trachea prior to intubation of a patient. In practice, however, the known drug delivery devices and procedures render a relatively uncontrolled delivery and, hence, relatively inaccurate dosing of the drug involved, easily leading to overdosing or under-dosing which are both detrimental.

It is inter alia an object of the present invention to provide a spray device and force control system that allows the physician to deliver a well controlled and more accurate administration of an active agent into the oral cavity of the patient, particularly to the patient's mouth, throat and/or respiratory tract.

To that end a spray device of the type described in the opening paragraph, according to the invention is characterized in that the mouthpiece is intended and arranged to be received between the lips of the user, that the inhalation member comprises a stop for receiving a dental element of the user, in that the mouthpiece comprises a spray cavity which, during use, opens into the oral cavity of the user, in that the spray unit opens into the spray cavity to deliver the mist via said spray cavity to the oral cavity of the user, particularly targeting the pharyngeal region.

The inhalation member according to the invention particularly comprises said stop that will rest against the denture of the patient to provide a predefined position within the oral cavity of the patient. The spray pressure and spray droplet size may then be chosen such that the generated mist will particularly target a particular area of the patient's upper airways, for instance the pharyngeal area of the throat of the patient. As a result substantially the entire dose that is being administered will actually land distally in the targeted area, allowing a more precise dosing and avoiding hyper-dosing or sub-dosing. Such stop may be configured in a wide variety of ways. A specific embodiment of the spray device according to the invention, however, is characterized in that the stop comprises an edge, such as a rib, shoulder or depression, on a circumference of the inhalation member.

The container may have a dosing mechanism that allows the physician to manually control the amount of liquid that is being delivered. From a practical perspective it is desirable for both the physician and the patient that such manipulation is being performed at a distance in order to render the procedure less intrusive for the patient and more ergonomic for the physician. The liquid line between the inhalation member and the container may comprise a flexible tube that allows for such remote operation. Thereby, a preferred embodiment of the spray device according to the invention is characterized in that the liquid line maintains a substantially fixed connection with the spray nozzle unit at a first end, that the liquid container is manually detachably coupled to a second end of the liquid line, in particular by means of a manually detachable coupling, more in particular a Luer lock coupling. This allows to use multiple containers without requiring the patient to remove the inhalation member.

During administration of the active agent the patient may be asked to inhale while a soft mist spray is being delivered in order to assist in targeting the more distal region of the oral cavity.

To that end, a specific embodiment of the spray device according to the invention is characterized in that the inhalation member comprises at least one air inlet which enables the user to inhale through the mouthpiece and directs an inhaled air stream via an airflow path to the spray cavity. In that case the spray may be delivered directly into an inhaled air stream to be carried along with that air stream to the distal region of the oral cavity or even further into the respiratory system of the patient. As such the spray device may also be employed for pulmonary delivery of a drug.

A certain flow resistance may be built into the device in order to balance the airspeed at which the inhaled air will pass the spray cavity. To that end a specific embodiment of the spray device according to the invention has the feature that said airflow path crosses an airflow resistance member. More particularly, the spray device according to the invention may further be characterized in that said airflow path crosses a filter member.

It is desirable to have a traverse airflow over the outlet of the spray nozzle in order to avoid that consecutive droplets of the soft mist that is being generated may coagulate with one another. To that end a particular embodiment of the spray device according to the invention is characterized in that the at least one air inlet is in open communication with a number of air channels that are distributed radially around an outlet of the spray nozzle unit and open in pairs toward one another.

The device according to the invention particularly may be a disposable device, intended for single use on a single patient only. To that end, a specific embodiment of the spray device according to the invention is characterized in that said container is a disposable medicinal syringe.

The spray device according to the invention may be used to deliver a controlled spray of an active agent or medicament for direct coverage of the oral cavity and pharynx.

The spray device according to the invention may be used to deliver a controlled spray for pulmonary administering of an active agent/medicament.

The spray device according to the invention may be supplied as an empty device; a medical professional chooses the medication and exact dose and volume.

The spray device according to the invention may be indicated to be used on patients that are deemed suitable for treatment by the medical professional.

In a specific embodiment the spray device according to the invention is used in conjunction with a liquid container comprising a liquid reservoir that debouches on a first side into said liquid line and on an opposite side comprises a plunger member which is sealingly displaceable therein, said plunger being configured and adapted to expel liquid from said container towards said spray nozzle under an elevated operating pressure. The spray device according to the invention may be adaptable to commercially available Luer-lock syringes.

The spray device according to the invention may particularly be used for the pharyngeal delivery of an anaesthetic agent. In order to particularly target the distal area of the oral and pharyngeal cavity, a particular embodiment of the spray device according to the invention is characterized in that said spray nozzle comprises a nozzle plate having a number of substantially identical nozzle orifices that receive said liquid and nebulize said liquid into droplets of approximately equal size, wherein said orifices have an effective diameter of at least 3.5 micrometre, particularly between 3.5 micrometre and 10 micrometre. In this respect the effective diameter of a spray orifice is defined to be the diameter of a perfect circle have a same surface area as a cross section of the orifice concerned. It turns out that nozzle orifices of the indicated size will deliver spray droplets of over 10 micrometre size that will typically land distally in the patient's pharyngeal cavity.

The spray device according to the invention may also be used for a pulmonary delivery of an active agent. In order to particularly target that area of the respiratory system of the patient, particularly the pulmonary region, a particular embodiment of the spray device according to the invention is characterized in that said spray nozzle comprises a nozzle plate having a number of substantially identical nozzle orifices that receive said liquid and nebulize said liquid into droplets of approximately equal size, wherein said orifices have an effective diameter of below 3.5 micrometre, particularly between 1 micrometre and 3.5 micrometre. In this respect the effective diameter of a spray orifice is defined to be the diameter of a perfect circle have a same surface area as a cross section of the orifice concerned. It turns out that nozzle orifices of the indicated size range will deliver spray droplets of less than 10 micrometre size that will typically pass the pharyngeal cavity of the patient to enter into the respiratory tract.

The present invention moreover relates to a force control system that may be used in conjunction with the spray device that is described hereinbefore and that will aid in manually delivering a substantially constant operating pressure. To that end, a force control system according to the invention, comprises a holder for accommodating an exchangeable product container, which product container comprises a reservoir holding a liquid to be nebulized that contains a therapeutic, in particular pharmaceutical, active agent for oral administration, particularly for pharyngeal or pulmonary administration.

The force control system further comprises a mouthpiece spatially separated from the product container, which is intended and adapted to be orally received by a user, said mouthpiece comprising a spray nozzle unit which is configured and adapted to receive said liquid to be nebulized under an elevated operating pressure and to dispense a mist therefrom containing the active agent. The force control system comprises a liquid line between the product container and the mouthpiece connecting said reservoir to said spray nozzle unit for a supply of said liquid.

The liquid reservoir debouches on a first side into said liquid line and on an opposite side comprises a plunger member which is sealingly displaceable therein, said plunger being configured and adapted to expel liquid from said container towards said mouthpiece under an elevated operating pressure.

The holder comprises a push member that engages directly or indirectly said plunger member, said push member being configured and adapted to impose said substantially constant operating pressure on said plunger, wherein said holder comprises at least one manipulator member that is configured and intended to transmit a manually exerted force by the user to said push member. The manipulator member is coupled to the push member via a power transmission that provides a force-increasing power transmission. The push member may engage the plunger member against a resilient spring tension.

Said power transmission may comprise a lever which is configured to allow first displacement that axially displaces the push member over a second displacement, wherein the first displacement is greater than the second displacement.

The manipulator member may comprise a squeeze handle extending from a housing of the holder, in that the squeeze handle is configured to allow a stroke against a spring tension relative to the housing that imposes said first displacement on the lever.

The transmission may comprise a intermediate device which is on the one hand resiliently coupled to the push member and on the other hand to the at least one manipulator member. The holder may comprise a housing with an axial body cavity that accommodates the intermediate device and the push member, wherein the manipulator member successively engages a shoulder of an axial series of shoulders extending from the intermediate device, and wherein said shoulders mutually have a substantially fixed pitch that defines an incremental stroke travel by the push member.

Hereinafter, the invention will be described in further detail with reference to a specific embodiment and an accompanying drawing. In the drawing:

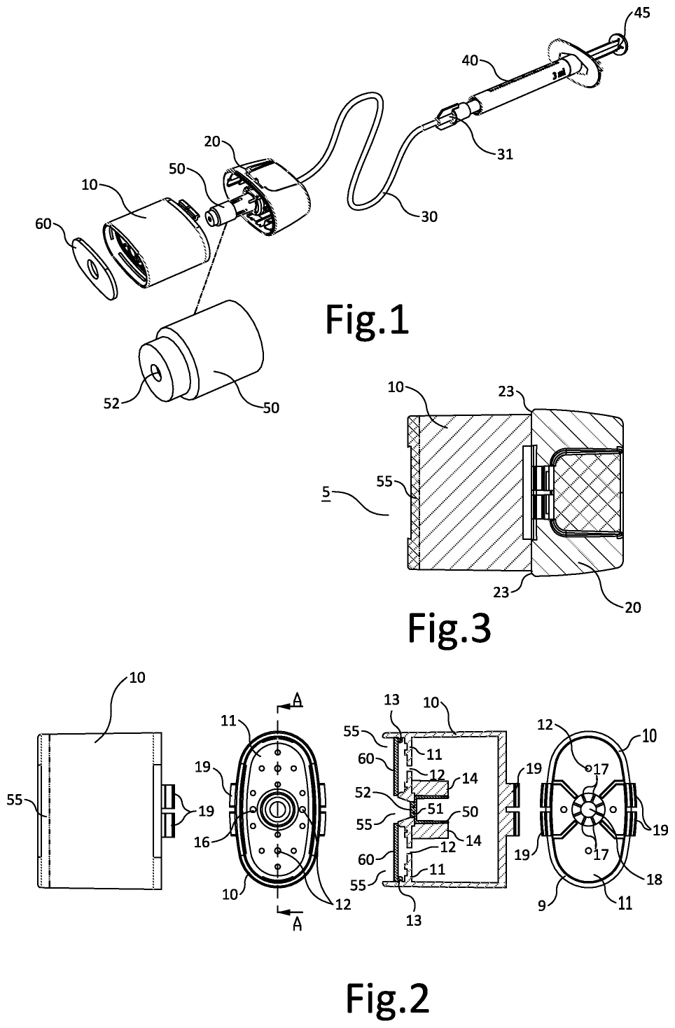

FIG. 1 is an exploded view of an example spray device according to the invention;

FIG. 2 is a detailed drawing of the mouth piece of the device of FIG. 1;

FIG. 3 is a side view of the inhalation member of the device of FIG. 1;

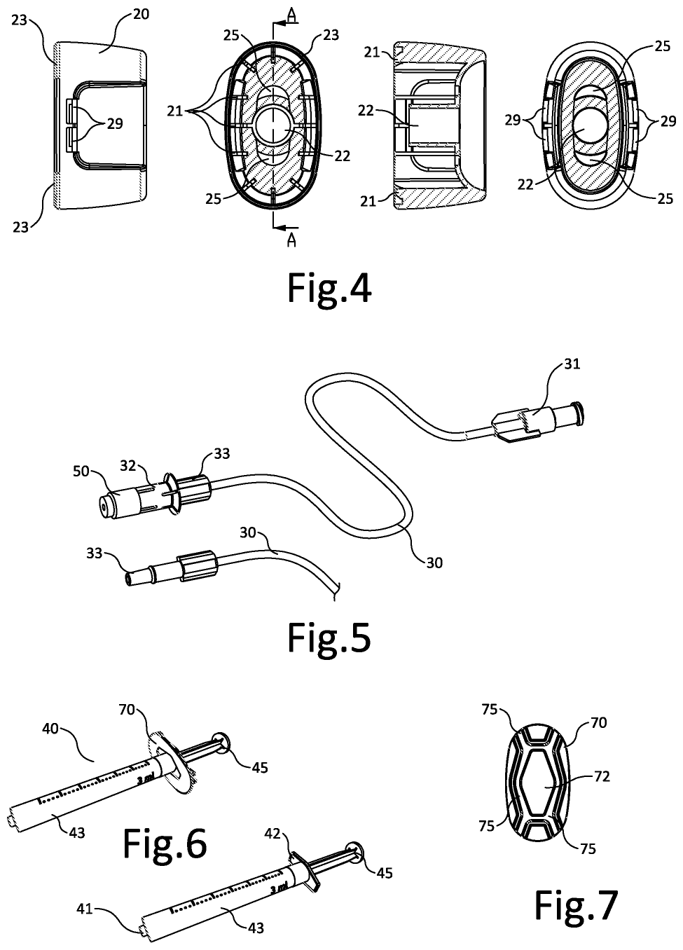

FIG. 4 is a detailed drawing of a base member of the device of FIG. 1;

FIG. 5 is a detailed drawing of the connection set of the device of FIG. 1, with and without a spray nozzle unit and spacer member;

FIG. 6 is a detailed drawing of a syringe to be used with the device of FIG. 1, with and without a comfort flange member;

FIG. 7 is a detailed drawing of the comfort flange member used with the syringe of FIG. 6;

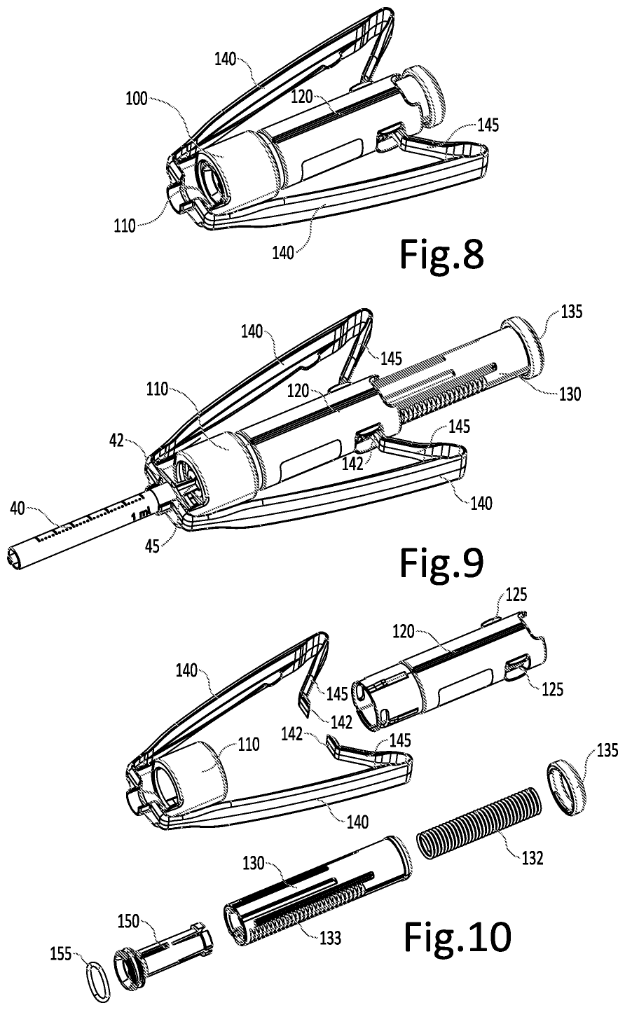

FIG. 8 is a perspective view of a holder of a force control system according to the invention;

FIG. 9 is a perspective view of the holder of FIG. 8, loaded with a medical syringe;

FIG. 10 is an exploded view of the holder of FIG. 8; and

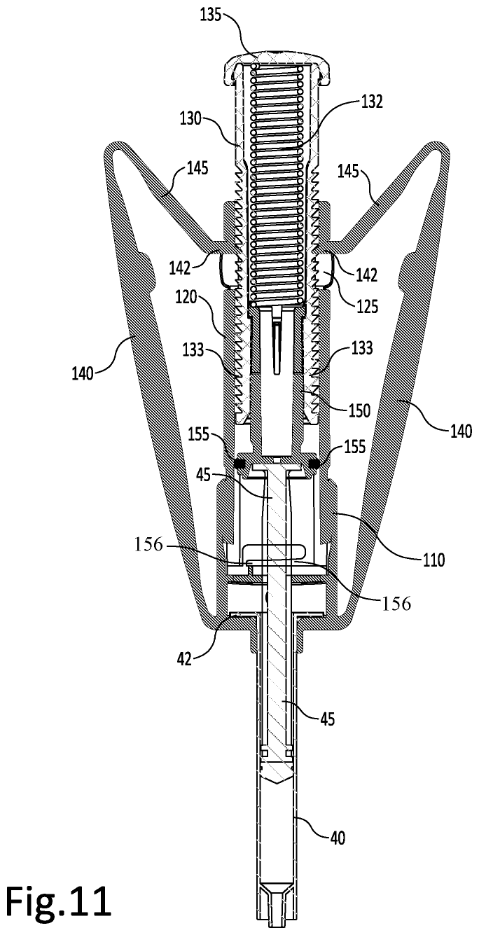

FIG. 11 shows a cross section of the holder of FIG. 8, loaded with a medical syringe.

It is noted that the figures are drawn purely schematically and not necessarily to a same scale. In particular, certain dimensions may have been exaggerated to a more or lesser extent to aid the clarity of any features.

An embodiment of the device according to the invention is shown in FIG. 1 and comprises an assembly of several functional parts:
An inhalation member 5, comprising a mouth piece 10, that is ergonomically shaped to be orally held between the lips and against the teeth of the patient for the controlled targeting of a spray to the patient's mouth and throat and a base member 20, that allows the patient to manually manipulate and hold the device;
A spray nozzle assembly, comprising an adapter body 50 holding a spray nozzle unit 51 with a spray nozzle 52 that delivers a liquid spray into a spray cavity 55 within the mouth piece 10.
A connection set: comprising a flexible connection tube 30 with Luer-lock connection 31 for connecting to a disposable Luer-lock syringe 40, particularly of 1 ml to 3 ml size, and a spray nozzle hub 33 for holding the spray nozzle assembly 50 and a locking member 32 for fixation of the spray nozzle hub 33 and spray nozzle assembly 50; and
A flow filter member 60.

The device according to the invention may be used for drugs that are approved for oral administration, particularly with the intention of pharyngeal or respiratory (tracheal and/or pulmonary) delivery. The device is preferably be used with a standard disposable syringe 40, typically 1 ml to 3 ml, that is provided with a Luer-lock connection. In a first embodiment, the syringe is filled with a 2% or 4% lidocaine solution (40 mg/ml) that is intended for pharyngeal delivery. Alternatively, the device may also be used for a pharyngeal delivery of another pharyngeal drug, like a chloroprocaine formulation.

The mouthpiece 10 is shown in FIG. 2 in greater detail and is intended for oral insertion into an adult patients mouth. From left to right, FIG. 2 shows the mouthpiece in side view, frontal view, cross section and rear view, respectively. It comprises a mainly hollow member that is formed out of a transparent bio-compatible polypropylene. The patient will be asked to close their lips around the mouthpiece. This member features a front wall 11 with a number of holes 12 that allow the patient to inhale through the mouth piece while imposing a certain airflow resistance. This resistance forces the patient to inhale slowly as the inhalation speed is maximized by the internal flow resistance. The added flow filter member 60 comprises a sheet filter 60 that is press fitted in a spray cavity 55 that extends within the mouthpiece 10. The flow filter 60 aids in creating an laminar airflow from the initially relatively turbulent flow as emanating from the front wall 11 holes.

The spray nozzle assembly 50 is placed in the centre of the front wall 11 and is held in position by a number of circumferentially distributed wings or ribs 14 that extend integrally from the rear face of the frontal wall 11. These four ribs 14 clamp around the spray nozzle assembly 50 and take care of a concentric alignment of the spray nozzle assembly and the mouthpiece 10 such that the spray nozzle 52 opens via a central hole 18 in the front wall 11 into a spray cavity 55 at the front of the mouthpiece 10. A number radially distributed grooves 17 are arranged along a circumference of said central hole 18. The central hole 18 provides a passage for the spray that is generated by the spray nozzle 52 of the spray nozzle unit 51, while the grooves 17 create opposite side flows that interact with spray droplets as they are released by the spray nozzle 52 with the purpose of preventing coagulation of the droplet trains (rays or jets) that are generated by the spray nozzle 52.

An internal wall of the spray chamber 55 has studs or a rim 13 that define(s) a distance between front wall 11 and flow filter member 60. The sheath flow filter 60 comprises a porous polyethylene body. The porous structure of the material creates a turbulent airflow from the air passing through the flow resistance wall of the mouth piece. The parts outer contour is manufactured with precision, so that it has a tight fit inside the mouth piece internal fixture. The centre hole shall fit tightly around the mouth piece flow resistance wall. The sheath flow filter is made from sintered sheet material. No loose particles are allowed, as the final device shall be free of small particles or dust.

Like the mouthpiece 10, also the base member 20 is made out of transparent polypropylene and is shown in greater detail in FIG. 4. From left to right, FIG. 4 shows the base member 20 in side view, frontal view, cross section and rear view, respectively. The base member 20 features a plurality of internal ribs 21 that support the mouthpiece 10 and prevent the mouthpiece assembly 10,20 from being compressed when in the assembled state. The ribs play 21 an important role for the realisation of a one-way snap with mouthpiece assembly. The mouth piece 10 and base member 20 shall form one solid part after assembly. Therefore a one-way-snap connection connects the two product halves 10,20. The snap fit 19,29 has a symmetrical design, so that it can be assembled upside down. The snap fit consists of a double set of snap fingers 19 and cavities 29 to prevent the product to be disassembled easily. The slanting profile of the ribs 21 at the inside of the base member 20 pushes the mouthpiece 10 snap fingers 19 outwards the outside when the snap is activated. This counteracts unintended disassembly of the two product halves 10,20. The base member 20 is given greater dimensions than the mouthpiece 10 in order that a circumferential rim 23 is formed at the interface between both parts. This rim 23 will serve as a stop for receiving a dental element of the user in order to obtain a predefined positioning of the mouthpiece 10 and spray nozzle 52 with respect to the oral cavity.

The base member 20 serves as a back stop for the connection set assembly 30-33 that connects to a disposable syringe 40. Said connection assembly comprises a flexible tube 30 with a spray nozzle unit hub 33 sealed at one end of said tube 30 and a Luer lock connector 31 at the opposite end, see also FIG. 5. A spacer member 32 slips over the nozzle hub and has a widened base flaring out that is supported by a rim of a central passage 22 in the base member 20. The nozzle hub 33 has an internal lumen that opens at en front end that has a little taper to press fitted in a corresponding conical internal cavity of the adapter member 50 of the spare nozzle unit 51 to establish a leak free connection.

A spacer member 32 is designed to snap over the male Luer tip of the nozzle hub member 33. It has an internal snap feature that creates a one-way snap action. Assembling the spacer member 32 on the connection set requires a stroke press set-up. This groove determines the rigidity of four concentric one-way-snap ribs. This connection set assembly 32,33 is inserted into the base member 20 and then pressed in the mouth-piece 10 with some pre-tension. The spacer member 32 generates this pre-tension with a slight deformation of the spring finger assembly 21. This pre-tension facilitates a hard contact between the spray nozzle unit 51,52 and the internal flow resistance wall 11 at the location of the pit flow holes 12. These four grooves determine the rigidity of the pre-tensioning feature, pushing the spray nozzle unit towards the pit-plane in the mouth piece.

As the device is inserted in the patient's mouth, the base member 20 serves as an indicator for the depth of insertion. The patient is asked to insert the device until the teeth touch a rim 23 that is provide by base member 20 since the base member 20 has slightly larger external dimensions than the mouthpiece 10 as shown in FIG. 3. The base member 20 is intended to be manipulated by the user (being the medical professional and the patient) as the mouth piece 10 is intended for mouth contact by the patient only.

When the patient is asked to take aspirations, the medical professional will push firmly on the plunger rod 45 of the syringe 45, thus emptying the active formulation into the patient's oropharyngeal cavities. The base member 20 has two half-moon shaped air inlets 25, that allow air to flow through the mouthpiece 10. These air inlets 25 have been placed in a recessed shape opposite of one another, making it impossible to accidentally block the airflow with the fingers. The spray nozzle unit 50-52 converts this pressurized liquid into a soft mist of droplets of predetermined size mainly depending on the size of the spray orifices of the spray nozzle unit. With the aim of targeting primarily the pharyngeal region the size of the orifices of the nozzle 52 is chosen such that they will release droplets having a substantially equal nominal size of between 10 and 20 micrometre. This size will promote the droplets to land particularly on the pharyngeal area of the patient.

The connection set 30-33 between the syringe 40, with the lidocaine formulation, and the mouthpiece 10 with the soft mist spray nozzle unit 51,52, allows the medical professional to focus on the act of pushing the syringe empty and allows the patient to focus on the act of inhalation through the mouth and exhaling through the nose. The connection set is shown in FIG. 5 in greater detail and facilitates the connection with any 1 ml to 3 ml syringe. The internal diameter (ID) of the connection set is preferably approximately 1 mm. A wider ID will cause unnecessary dead volume of the system and spillage of formulation. A smaller ID will influence the flow resistance of the system.

A male Luer connection at the end of the nozzle hub 33 connects leak free to a female Luer adapter member 50 of the spray nozzle assembly 50-51. The other end of this set is equipped with female Luer-lock connector 31. The syringe 40 has a male Luer-lock connection that fits to this female part 31 that is connected to the free end of the connection tube 30. For disconnecting a connected empty syringe, the user must apply a certain torque. This torque is easier to generate with a fin that is provided on the connector. For successful spray generation, it is important to generate a hydrostatic pressure exceeding 7 bar. All connections withstand this pressure without any deformation or leakage.

FIG. 5 shows the completely assembled connection set, comprising the spacer member 32 and spray nozzle unit 50. This set connects to the disposable syringe 40, particularly a standard 1 ml to 3 ml disposable syringe with male Luer-lock connector 41 and plunger rod 45. An example of a standard syringe 40 is shown in FIG. 6 as it is commercially available (bottom) as well as it may be used in combination with a novel comfort flange member 70 (upper). As it is required that the medical professional applies a significant force during approximately 1 minute, resulting in about 7 bar spraying pressure, only a 1 ml to 3 ml syringe is advised. Larger syringe volumes feature larger plunger diameters and, hence, demand higher actuating forces by the user.

The comfort flange member 70, as depicted in greater detail in FIG. 7, makes it easier to apply the required force to the syringe plunger rod 45. The comfort flange 70 has been designed to fit over most available disposable syringes, from 1 ml to 3 ml. A recess 72 with a thinner shape fits closely the syringe native flange 42, so that the comfort flange 70 is oriented correctly with respect to the syringe 40. It is placed around the flexible tube 30 and can be shifted over the syringe barrel 43 after the syringe 40 has been connected to tube connector 31. The flange 70 is packaged with the connection set in order to make it more logical for the user to shift it over the syringe 40 after connecting the syringe to the connection set. The flange member 70 is provided with a number of groove channels 75 that can accommodate and hold in place the flexible tube 30 while being packaged with the connection set 30-33.

In practice, larger syringes and, particularly, smaller droplets of the mist to be created, in practice, lead to an increase of the forces required to impose the necessary operating pressures as the internal resistance of the air spray device thereby generally increases. In order to aid the physician to exert such an increased force, the present invention also provides for a force control system of the kind as depicted in FIGS. 8-11. The system comprises a holder 100 as shown in FIG. 8 that may receive and accommodate a standard disposable syringe 40 as is shown in FIG. 9. This part features a bay 110 at the front to hold the syringe barrel 43, including the flange 42 and plunger rod 45 of the syringe. The syringe barrel 43 is enclosed by slightly more than half the circumference of the barrel, snapping the syringe barrel in place by the material's elasticity of the holder 100 wall. The top side of the flange 42 of the syringe barrel can be pressed against a wall and avoids turning of syringe barrel along its axis, since the flange has no full freedom of rotation. An insertion bay 110 at one side of the holder 100 provides enough space to accept the insertion of a full syringe (with the plunger rod fully extended).

The holder 100 comprises a cylindrical housing 120 that accommodates an intermediate member 130 as part of a transmission between a set of outwardly flaring handles 140 and a push member 150 that engages the plunger rod 45 that connects to the plunger of the syringe 40. In between the handles 140 and the intermediate member 130 are levers 145 that will increase the force that is transmitted to the push member 150 as compared to the force that is manually exerted by means of the handles 140. The handles 140 are for the user to hold and squeeze with a single hand. The levers 145 accept the manual force applied by the user over a certain distance and transmit the force over a shorter distance (amplifying the force) in the axial direction of the syringe.

The push member 150 is slidingly received inside a cavity of the intermediate member 130 against the action of a coil spring 132 that resides within the intermediate member. An end cap 135 closes said member 130 at the rear end and compresses said spring 132 between said end cap 135 and said push member 150. The levers 145 are designed to force the intermediate 130 member traveling such a distance that the spring 132 is compressed enough to allow for a proper dispensing travel and time. This travel is designed to be 6 mm in the present case. The depicted setup decouples the manual force that is exerted on the handles 140 with respect to the force that is actually imposed by the push member 150 on the plunger rod 45 of the syringe 40 in order to more evenly distribute said force over a full compression stroke by the handles 140. This delivers a constant and controlled operating pressure at the output orifice of the syringe independent of the compression force that is exerted by the user.

Each of the levers 145 carry a tooth 142 at its distal extreme end that extends through respective windows 125 in the outside wall of housing 120 of the device to engage a respective racked gear 133 that is formed in the outside wall of the intermediate member 130, as particularly shown in the cross section of FIG. 11. The racked gears 133 have a regular pitch that defines an incremental stroke length of the intermediate member 130. Upon each full squeeze of the handles 140 by the user, a total stroke is determined by the axial travel of the teeth 142 that is transmitted to the intermediate member 130 and the push member 150 via the windows 125. This stroke is transmitted via the push member 150 to the plunger rod 45 of the syringe and, hence, translates to a controlled, predetermined dosage of liquid that is being expelled from the barrel 43 of the syringe. The friction by a resilient O-ring 155 maintains the intermediate member 130 in position once the handles 140 are released in order that the teeth 142 will engage a next shoulder of the racked gear 133.

The depicted force control system provides a device to actuate syringes that may part of the spray device of the present invention, as described hereinbefore, that may for instance be used for pharyngeal, tracheal or pulmonary delivery of an active agent, which may require a relatively high force. To comfortably apply and distribute such force, a medical professional manually compresses the set of two handles 140 and levers 145, which in turn move the ratchet 133 by one stroke. This ratchet 133 presses onto the syringe's plunger through the intermediate of the pre-compressed spring 132, evenly distribution the force until the either the spring reaches the end of its allowed travel or the user releases its force on the levers 140,145. The device accepts 1 ml and 3 ml Luer-lock syringes and has a pressure relief system to allow expansion of the syringe plunger.

Furthermore, the force control system also has a mechanism to allow expansion of the syringe plunger, preventing unwanted drooling or dripping of the spray device as soon as the user stops actuating the system: Upon full compression of the Lever handles 140, the lever teeth 142 push the intermediate member 130 forwards with a predefined stroke. In the first few millimetres of this stroke, the two leaf springs 156 that are situated on the distal end of the housing 120 are being compressed as the housing 120 is pushed forward by the interconnection between push member 150 and its o-ring 155 and the housing 120. The housing 120 slides in to the lever part 140 for a length of approximately 2 mm. After compression of these leaf springs 156, the pusher spring 132 is now compressed for a predefined stroke, resulting into a direct and constant actuating force, driving the push member 150 and the plunger rod 45 forward and driving the formulation out of the syringe 40 into the spray device. As soon as the user releases the compression of the lever handles 140, it is important that the actuation force coming from the compressed spring 132 is instantly removed so that and any axial compression in the syringe and connection set is removed, leaving no hydraulic pressure in the system causing unwanted dripping or drooling.

When the lever handles 140 are released, the o-ring 155, functioning as a friction element between housing 120 and push member 150 holds its position. The spring 132 will therefore regain its full length bringing the intermediate member 130 backwards. At the same time, the two leaf springs 156 will regain their relaxed position of approximately 2 mm between the distal end of the levers part 140, where the syringe barrel 42 is positioned and push member 150. This 2 millimetre extra space is therefore created between plunger rod 45 and push member 150, leaving no residual pressure on the plunger rod 45.

Although the invention is herewith described with reference to this embodiment as shown in the figures it will be appreciated that the invention is by no means limited to that embodiment. On the contrary, many more embodiments and variation to the invention are feasible to a skilled person within the scope of the present invention.

1. Spray device for the oral administration of a therapeutic, in particular pharmaceutical, active agent, comprising a liquid container for holding an amount of a liquid to be nebulized that comprises the active agent and comprising an inhalation member, comprising a mouthpiece that is intended and adapted to be received orally by a user, wherein said inhalation member is spatially separated from the container and is coupled to the container via a flexible fluid line, wherein said mouthpiece comprises a spray nozzle unit which is configured and adapted to receive the liquid to be atomized under an elevated operating pressure and to create a mist therefrom containing the active agent, wherein the mouthpiece is intended and arranged to be received between the lips of the user, wherein the inhalation member comprises a stop for receiving a dental element of the user, wherein the mouthpiece comprises a spray cavity which opens at a first end, and wherein the spray unit opens at a second end into the spray cavity to deliver the mist via said spray cavity to the oral cavity of the user, particularly targeting the pharynxial region.

2. Spray device according to claim 1, wherein the liquid line maintains a substantially fixed connection with the spray nozzle unit at a first end, wherein the liquid container is manually detachably coupled to a second end of the liquid line, in particular by means of a manually detachable coupling, more in particular a Luer coupling.

3. Spray device according to claim 1, wherein the stop comprises an edge, such as a rib, shoulder or depression, on a circumference of the inhalation member.

4. Spray device according to claim 1, wherein the inhalation member comprises at least one air inlet which enables the user to inhale through the mouthpiece and directs an inhaled air stream via an airflow path to the spray cavity.

5. Spray device according to claim 4, wherein said air flow path crosses an air flow resistance member.

6. Spray device as claimed in claim 4, wherein that said air flow path crosses a filter member.

7. Spray device according to claim 4, wherein the at least one air inlet is in open communication with a number of air channels that are distributed radially around an outlet of the spray nozzle unit and open in pairs toward one another.

8. Spray device according to claim 1, wherein said container is a disposable medicinal syringe.

9. Spray device according to claim 1, wherein the liquid line comprises a flexible hose.

10. Spray device according to claim 1, wherein the container comprises a movable plunger member on a side remote from an outlet to the liquid line that transmits said elevated operating pressure to the liquid.

11. Spray device according to claim 1, wherein said spray nozzle comprises a nozzle plate having a number of substantially identical nozzle orifices that receive said liquid and nebulize said liquid into droplets of approximately equal size, wherein said orifices have an effective diameter of at least 3 micron, particularly between 5 micron and 10 micron.

12. Spray device according to claim 1, wherein said spray nozzle comprises a nozzle plate having a number of substantially identical nozzle orifices that receive said liquid and nebulize said liquid into droplets of approximately equal size, wherein said orifices have an effective diameter of below 5 micron, particularly between 1 micron and 3 micron.

13. Spray delivery system, comprising:
a holder for accommodating an exchangeable product container, which product container comprises a reservoir holding a liquid to be nebulized that contains a therapeutic, in particular pharmaceutical, active agent for oral administration;
a mouthpiece spatially separated from the product container, which is intended and adapted to be orally received by a user, said mouthpiece comprising a spray nozzle which is configured and adapted to receive said liquid to be nebulized under an elevated operating pressure and to dispense a mist therefrom containing the active agent; and
a liquid line between the product container and the mouthpiece connecting said reservoir to said spray nozzle for a supply of said liquid;
wherein the liquid reservoir debouches on a first side into said liquid line and on an opposite side comprises a plunger member which is sealingly displaceable therein, said plunger being configured and adapted to expel liquid from said reservoir towards said spray nozzle under an elevated operating pressure;
wherein said holder comprises a push member that engages directly or indirectly said plunger member, said push member being configured and adapted to impose said elevated operating pressure on said plunger;
wherein said holder comprises at least one manipulator member that is configured and intended to transmit a manual force by the user to said push member; and
wherein said manipulator member is coupled to the push member via a power transmission that provides a force-increasing power transmission.

14. Spray delivery system according to claim 13, wherein said power transmission comprises a lever which is configured to allow first displacement that axially displaces the push member over a second displacement, wherein the first displacement is greater than the second displacement.

15. Spray delivery system according to claim 14, wherein the manipulator member comprises a squeeze handle extending from a housing of the holder, wherein the squeeze handle is configured to allow a stroke against a spring tension relative to the housing that imposes said first displacement on the lever.

16. Spray delivery system according to claim 13, wherein the transmission comprises a spring device which is on the one hand resiliently coupled to the push member and on the other hand to the at least one manipulator member.

17. Spray delivery system according to claim 16, wherein the holder comprises a housing with an axial body cavity that accommodates the spring device and the push member, and wherein the manipulator member successively engages a shoulder of an axial series of shoulders extending from the spring device, wherein said shoulders mutually have a substantially fixed pitch that defines an incremental axial stroke by the push member.

18. Spray delivery system according to claim 13, wherein the push member engages the plunger member against a resilient spring tension.

19. Spray delivery system according to claim 13, wherein the product container comprises a disposable medicinal syringe.

20. Spray delivery system according to claim 13, comprising the spray device according to claim 1.
